(19) United States
(12) Patent Application Publication
(10) Pub. No.: US 2019/0261952 A1
(43) Pub. Date: Aug. 29, 2019

(54) OPTIMIZATION IN ULTRASOUND COLOR FLOW IMAGING

(71) Applicant: Siemens Medical Solutions USA, Inc., Malvern, PA (US)

(72) Inventors: Paul Donald Freiburger, Seattle, WA (US); Chengzong Han, Bellevue, WA (US); King Yuen Wong, Issaquah, WA (US)

(21) Appl. No.: 16/217,083

(22) Filed: Dec. 12, 2018

Related U.S. Application Data
(60) Provisional application No. 62/636,077, filed on Feb. 27, 2018.

(51) Int. Cl. A61B 8/08; A61B 8/14

(52) U.S. Cl. CPC A61B 8/488; A61B 8/483; A61B 8/463; A61B 8/14; A61B 8/5246

(57) ABSTRACT

Rather than trying to automate what an experienced user does, rules designed for processor implementation are used for color flow imaging optimization by an image processor of an ultrasound scanner. By determining a characteristic of a scanned target, a priori information is provided. This a priori information, such as a size of a primary target, is used to select the optimization to be used. Different types of optimization may be used for different characteristics of the primary target. The values for settings may be different for different characteristics.

### RELATED APPLICATION

The present patent document claims the benefit of the filing date under 35 U.S.C. § 119(e) of Provisional U.S. Patent Application Ser. No. 62/636,077, filed Feb. 27, 2018, which is hereby incorporated by reference.

### BACKGROUND

The present embodiments relate to ultrasound-based color flow imaging. Color flow imaging estimates the velocity, power or energy, and/or variance of motion in a patient from ultrasound return echoes. The user sets a velocity scale, a gain, transmit frequency, temporal persistence, and/or parameters to configure the color flow imaging. Color flow optimization in ultrasound imaging is a user intensive activity that requires a fair amount of skill to properly generate good images for different targets and body types. It may be time consuming and challenging to find a satisfactory combination of settings for color flow imaging a particular patient.

Attempts have been made to automate optimization. The color flow signal may be analyzed to unwrap the phase or set the scale to prevent aliasing. A histogram may be used to estimate the maximum velocity for setting the scale. The gain may be set based on an estimate of the signal-to-noise ratio. Different targets and/or patients may not respond well to these automated optimizations. These approaches do not perform well because a “one size fits all” approach to color flow optimization is not robust. The optimization may not optimize well for different types of targets.

### SUMMARY

By way of introduction, the preferred embodiments described below include a method, system, computer readable medium, and instructions for optimizing color flow imaging. Rather than trying to automate what an experienced user does, rules designed for processor implementation are used for optimization by an image processor of an ultrasound scanner. By determining a characteristic of a scanned target, a priori information is provided. This a priori information, such as a size of a primary target, is used to select the optimization to be used. Different types of optimization may be used for different characteristics of the primary target. The values for settings may be different for different characteristics.

In a first aspect, a method is provided for optimizing color flow imaging by an ultrasound scanner. The ultrasound scanner scans a patient. A first color flow target represented in scan data from the scanning is segmented. A size of the first color flow target is determined. A scale and/or gain is set using a different criterion depending on the size of the first color flow target. The ultrasound scanner performs color flow imaging of fluid in the patient using the scale and/or gain.

In a second aspect, a method is provided for optimizing color flow imaging by an ultrasound scanner. The ultrasound scanner scans a patient. A first color flow target represented in scan data from the scanning is segmented. A value for a color flow imaging parameter is set based on a first characteristic of the first color flow target as segmented. Fluid in the patient is color flow imaged using the value for the color flow imaging parameter.

In a third aspect, an ultrasound system is provided for optimizing flow imaging. A transducer and beamformer are provided for scanning a scan region. A Doppler estimator is configured to estimate, from the scanning, flow values in the scan region. The configuration of the Doppler estimator is based on a detected size of a flow region in the scan region. A display is configured to display an image using the flow values.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart of one embodiment of a method for optimizing color flow imaging by an ultrasound scanner;

FIG. 2 is a flow chart of another embodiment of a method for optimizing color flow imaging by an ultrasound scanner;

FIGS. 3 and 4 are example color flow images generated with different gains based on target size; and

FIG. 5 is a block diagram of one embodiment of a system for optimizing flow imaging.

### DETAILED DESCRIPTION OF THE DRAWINGS AND PRESENTLY PREFERRED EMBODIMENTS

Ultrasound color flow imaging is automatically optimized. The scale, gain, persistence, transmit frequency, and/or another parameter are optimized based on a priori information for the patient being imaged. The scale, gain, persistence, transmit frequency are some of the most important parameters for color flow image quality optimization, and automatic optimization may reduce examination time and improve results and consistency. The automatic optimization problem is solved by using a priori information about the target(s) of interest to guide the optimization approach. A priori information is obtained through image segmentation and characterization of segmented targets to guide the automatic optimization. Different color targets need different optimization strategies. For example, automatically setting the scale based on the maximum velocity will kill sensitivity in small targets like signals in the testis, so scale may be set based on reduction of scale while avoiding flash for small targets and aliasing for large targets. As another example, the persistence is automatically optimized based on size or pulsatility. The persistence is increased for large and/or pulsatile targets and decreased for small and/or low pulsatility targets. The type of optimization is based on the segmented target for the patient.

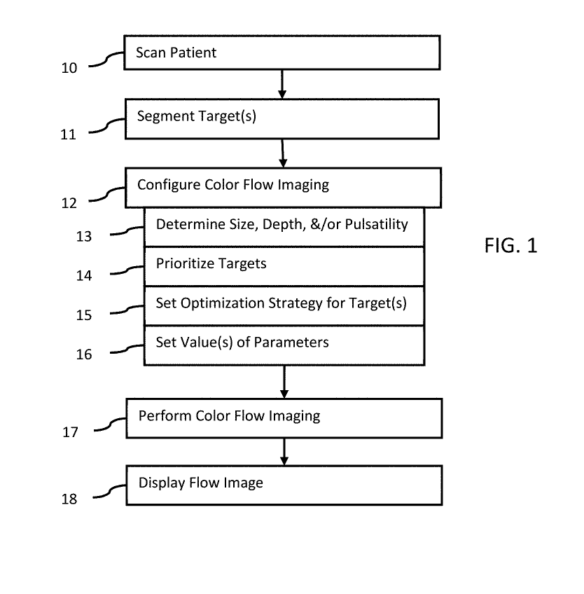

FIG. 1 shows one embodiment of a method for optimizing color flow imaging by an ultrasound scanner. Color flow is used to indicate spatial motion imaging, such as fluid or tissue motion. “Color” is used to distinguish from spectral Doppler imaging, where the power spectrum for a range gate is estimated. The estimated flow for each location is mapped to color for display, providing a spatial representation of motion in a scan region. The color “flow” data may not be of fluid (e.g., may be of tissue motion) and/or may not represent color (e.g., may be a scalar). Doppler, color, or flow imaging modes provide color flow imaging.

The method of FIG. 1 provides for optimization of the scale, gain, transmit frequency, persistence, and/or another type of parameter settings in color flow imaging. A priori information about one or more targets in a scan region of a patient are used to guide the type of optimization and/or the optimization. The values for settings and/or the strategy used to determine the values for settings are determined using, at least in part, the a priori information.

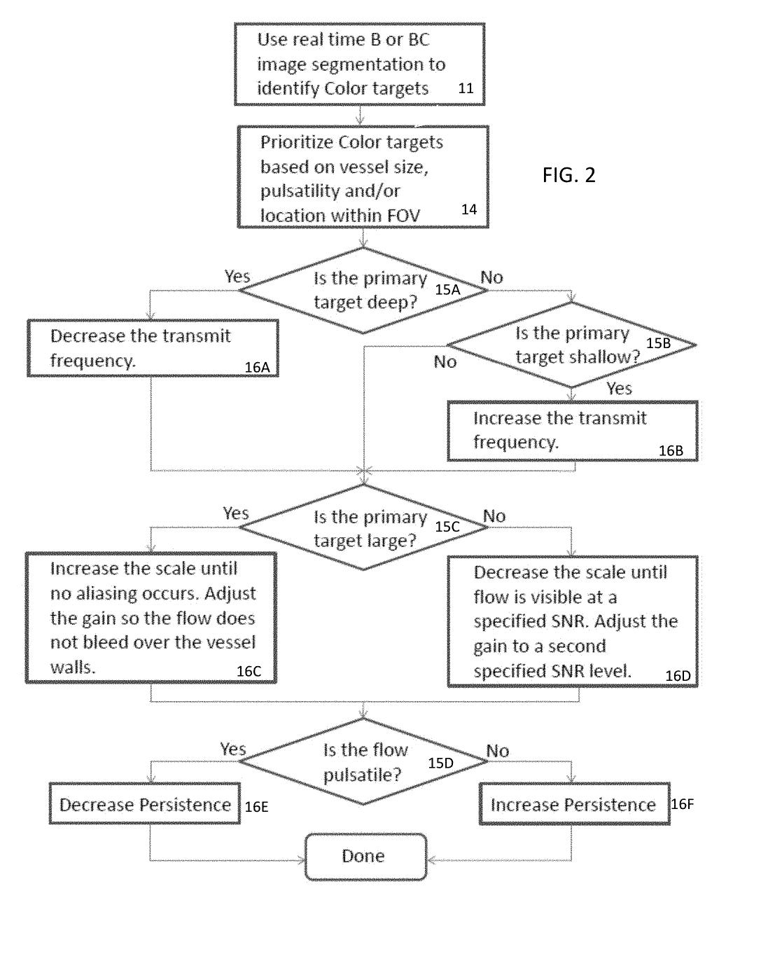

FIG. 2 shows a flow chart of another embodiment of the method for optimizing color flow imaging by an ultrasound scanner. An image processor performs the acts of the method to optimize the transmit frequency, scale, gain, and persistence in an example sequence.

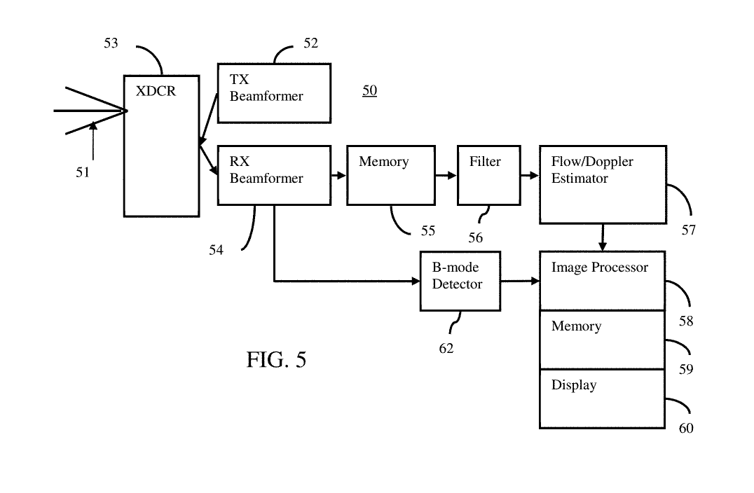

The methods of FIGS. 1 and 2 are performed by the ultrasound imaging system 50 of FIG. 5, the image processor 58, or a different system and/or processor. For example, the ultrasound imaging system 50 performs the acts. As another example, the image processor 58 controls a beamformer for scanning in act 10, segments in act 11, and configures a Doppler estimator in act 12. The Doppler estimator and a wall filter perform color flow imaging in act 17 using a beamformer and transducer of the ultrasound scanner. A display is used to display a color flow image after mapping motion scalar values from the Doppler estimator to color values. A scan converter, graphics memory, temporal filter, and/or other components of an ultrasound scanner may be used for any of the acts.

The acts of FIGS. 1 and 2 are performed in the order shown or a different order. For example, act 10 is repeated as part of act 12 and/or act 17. In another example, act 18 is provided as part of act 10 or 11. As another example and as shown in FIG. 2, acts 15 and 16 may be repeated to optimize different color flow imaging parameters. The color flow imaging parameters are any variable or setting available to control color flow imaging, such as scale, gain, transmit frequency, and/or persistence.

Additional, different, or fewer acts than shown in FIG. 1 or 2 may be used. For example, act 14 is not provided where a single target is designated and/or where characteristics from all the targets are combined. As another example, act 18 is not performed. In yet another example, acts for user adjustment, original setting, or manual control over the same or different color flow imaging parameters are provided.

In act 10, the ultrasound scanner scans a patient. Various locations within a scan region of the patient are scanned with ultrasound. In one embodiment using an ultrasound system, a field of view in a patient is scanned in real-time, providing images while scanning. The scanned region is an interior of an object, such as the patient. The scan is of a volume, plane, or line region. Scanning a plane provides data representing different locations or samples of the plane. The data representing the region is formed from spatial sampling of the object. The spatial samples are for locations distributed in an acoustic sampling grid.

The region for the color flow scan is a region of interest smaller than a field of view or for the entire field of view. The ultrasound system may scan the field of view using B-mode imaging, a combination of B-mode imaging and color flow imaging, or other modes of imaging. The color flow region is a sub-set of the B-mode field of view. The user or a processor determines the region of interest in which color flow scanning occurs. Alternatively, the color flow region is the full field of view.

Spatial samples along one or more scan lines are received. Where the transmit beam insonifies just one receive scan line, then return or echo samples along that scan line are received. Where the transmit beam insonifies multiple scan lines, then samples along the multiple scan lines may be received. To generate the samples for different receive beams at a same time, parallel receive beamformation is performed. For example, a system may be capable of forming two or more, tens, or hundreds of receive beams in parallel. Spatial samples are acquired for a plurality of receive lines in the region of interest in response to one and/or in response to sequential transmit beams.

The scanning may be performed a plurality of times to cover the region. The acts are repeated to scan different portions of the region of interest. Alternatively, performing once acquires the data for the entire region of interest.

For B-mode scanning, the scanning is configured to scan the field of view. For color flow scanning, scan lines in the region of interest are sampled multiple times. The complete region of interest is scanned multiple times in sequence. Scanning at different times in sequence acquires spatial samples associated with motion. Any now known or later developed pulse sequences and/or scan formats may be used for B-mode and color flow scanning. A sequence of at least two (flow sample count) transmissions is provided along each scan line for color flow imaging. For example, the flow sample count is 10-20, resulting in 10-20 samples for each location. Any pulse repetition frequency (i.e., rate of sampling for a location), flow sample count (i.e., number of samples for a location or used to estimate), and pulse repetition interval (i.e., time between each sample acquisition for a location) may be used. Only one transmission along each line is needed for B-mode imaging of the field of view for a given period.

The echo responses to the transmissions or return samples are used to determine intensity for B-mode scanning and estimate velocity, energy (power), and/or variance at a given time for color flow imaging. The transmissions along one line(s) may be interleaved with transmissions along another line(s). With or without interleaving, the spatial samples for a given time are acquired using transmissions from different times. The estimates from different scan lines may be acquired sequentially, but rapidly enough to represent a same time from a user perspective. Multiple scans are performed to acquire estimates for different times.

In alternative embodiments, the return samples (e.g., B-mode data and/or color flow data) are acquired by transfer over a network and/or loading from memory. Data previously acquired by scanning is acquired.

In act 11, the image processor segments one or more color flow targets represented in scan data from the scanning. The targets may be represented in B-mode information, such as identifying the targets as blood or fluid regions with less intensity, identifying tissue (e.g., vessel walls) that hold fluid, or identifying tissue that moves. Each target is a contiguous region of locations. Only one or more than one target may be identified, such as identifying one or more separate vessels represented in a scan plane. Each color flow target is separately segmented or identified as separate from other targets.

The targets may be represented in color flow information, such as identifying moving fluid or tissue. B-mode alone, color flow alone, or both B-mode and color flow data may be used to identify the target or targets.

The targets are in the region of interest for color flow imaging. The targets may be in a box or other designator of a region of interest less than the field of view or may be in the field of view. The targets are vessels, heart chambers, bladder, other fluid region, or tissue subjected to physiological cyclical motion. A boundary or mask region may be identified, such as vessel walls and/or fluid region between vessel walls.

Any segmentation may be used. For example, a threshold is applied to identify one or more fluid regions. The threshold may be applied to intensity, motion (e.g., velocity or power), or a gradient. In another example, region growing, skeletonization, random walker, template fitting, pattern matching, level set, fast marching, and/or other image processing is applied to identify or segment the targets. In yet another example, a machine-learned network outputs the segmentation in response to input of the scan data.

In one embodiment, the segmentation involves multiple processes, including preprocessing, segmentation or detection, and postprocessing. The preprocessing reduces background noise using linear and/or nonlinear filters (e.g., morphological filters like top-hat transformation or some other filter). The segmentation or detection applies a thresholding approach on power data and/or velocity data from color flow scanning. The threshold is predetermined as fixed value (e.g., 0.1 of maximum value) or could be determined based on automatical thresholding approaches (e.g., Otsu's method). The postprocessing step applies a connected-component algorithm to list all detected flow objects that are geometrically not connected after segmentation. The boundaries may be filtered to reduce variation along the edges. A size threshold may be applied to keep only targets larger than the threshold size. The geometrical characters of each flow object, such as area and shape, may be calculated and used to identify or not the targets.

The segmentation outputs one or more color flow targets. Each segmentation designates the boundary and/or locations belonging to the target. This segmentation is a priori information to guide the automatic optimization. The a priori information is obtained through image segmentation and/or characterization of B-mode and/or color flow mode image. The patient or scan specific a priori information about potential color flow targets is used to improve automatic color flow optimization.

In act 12, the image processor configures the ultrasound scanner (e.g., transmit beamformer, receive beamformer, wall filter, Doppler estimator, color mapping memory or processor, amplifier, temporal filter, and/or other component) for color flow imaging. One or more values of color flow imaging parameters are set. The color flow imaging parameter is any variable or setting used to control operation of the ultrasound scanner for color flow imaging. Example color flow imaging parameters include transmit frequency, scale, gain, and/or persistence.

The value of at least one setting for the color flow configuration is set based on one or more characteristics of the target or targets as segmented. Any characteristic may be used, such as the size (e.g., diameter, radius, area, volume, and/or distance), location in the field of view (e.g., depth and/or distance from the center), and/or pulsatility (e.g., variance in flow over a cycle or averaged over cycles). In act 13, the size, depth, and/or pulsatility are determined for each target. For example, the diameter of the vessel (e.g., longest distance between boundaries) is determined for each target. Other characteristics may be determined.

The characteristic is determined from the segmentation. The segmentation provides location relative to the field of view, such as location of a center and/or other spatial sample position relative to depth in or a center of the field of view. The segmentation provides the size, such as the longest diameter for a planar cross-section or arbitrary imaging slice through a vessel. The segmentation provides the pulsatility by indicating the locations of flow, for which the maximum and minimum (e.g., maximum and minimum of average flow or maximum and minimum over time of the maximum flow in the target) is calculated.

In act 14, the image processor prioritizes between targets. Where more than one target is identified in the segmentation, the targets may be prioritized. The prioritization selects one target or provides a relative ranking of multiple targets. The ranking allows for optimization that may vary by location or target verses optimizing for the primary or selected target. The selection provides for optimization to the selected target. The configuration is based on the selected target and/or top ranked targets.

The prioritization uses one or more determined characteristics of the target as segmented. For example, the relative size, pulsatility, and/or location in a field of view of the scanning is used. For example, the size is used to select (e.g., select the largest or smallest, such as smallest above a threshold size). As another example, the priority may be based on the pulsatility, such as ranking by most or least pulsatility. In another example, the location of the target relative to a center of the field of view is used to prioritize, such as selecting the target closest to the center of the field of view.

Combinations of characteristics may be used. Example combinations may include fuzzy logic or weighted average of ranks from different characteristics. One example combination is finding all the targets over a threshold size and then ranking those targets based on pulsatility and/or closeness to a center of the field of view.

The characteristic or combination of characteristics used to prioritize may depend on the application. The prioritization may be different for imaging the carotid artery than for imaging the thyroid. For thyroid imaging, small vessels and/or vessels with less pulsatility may be of interest. For carotid artery imaging, the largest vessel and/or most pulsatile vessel is of interest.

The prioritization may separate the identified targets into primary and secondary targets based on vessel diameter, pulsatility and/or distance from the center of the field of view. Color flow imaging parameters that may affect the prioritization include color flow wall filter threshold setting, scale, vessel boundaries, flash suppression, spatial filters, wall filters, frequency, and line density. These settings may be considered when prioritizing.

Once prioritized, the same or different characteristics of the selected target or targets (e.g., higher ranked or primary targets) are used to optimize. For example, the primary target(s) are characterized as large or small, pulsatile or not, and/or deep or shallow.

In act 15, the image processor sets the optimization strategy. Different strategies may be available for different types of targets. The algorithm to be used for optimization is selected where different algorithms use different criteria (e.g., weightings, functions, measures, orders, and/or operations), resulting in different strategies in optimizing color flow imaging. For example, measures of flash or aliasing are used to optimize scale. As another example, measures of noise (e.g., signal-to-noise ratio) or bleeding are used to optimize gain. The measure to be used is selected as the optimization strategy. In alternative embodiments, the selection of the strategy is whether to increase or decrease values of a setting (i.e., color flow imaging parameter). The same criterion may be used, but the direction of the adjustments in optimizing the parameter is selected. Any characteristics of the optimization may be selected to determine the optimization approach.

The optimization strategy is selected based on one or more characteristics of one or more targets. The a priori information from segmentation is used to determine the strategy. The same or different characteristics of the target used for prioritizing are used to selecting the strategy. For example, the depth in the field of view, pulsatility, and/or size of the target are used.

The optimization strategy is selected for one or more of the targets. The strategy may be selected based on the selected or primary target. The strategy may be selected based on multiple targets, such as using an average characteristic of the targets or selecting different optimization for different targets or locations.

In act 16, the selected optimization strategy is applied. One or more values for a respective one or more color flow imaging parameters are determined using the selected optimization strategy or strategies. The algorithm (e.g., operations or measures) is performed to optimize the color flow imaging parameter or parameters for imaging the specific patient and/or scan region (i.e., field of view or region of interest for color flow). The value is determined by iteratively testing different values adjusted based on feedback using the selected criterion or criteria. Other optimization approaches to determine a value may be used.

The application configures the ultrasound scanner for color flow imaging. One or more values may be defaults for the application and/or manually set by the user. One or more values are set by the selected optimization.

The strategy selection and application of acts 15 and 16 may be repeated for different color flow imaging parameters. For example, a transmit frequency strategy is selected, and then the value set. Then, the scale and/or gain strategy is selected and the corresponding values set. Finally, the persistence strategy is selected, and the corresponding values set. Other orders, additional or fewer color flow imaging parameters, and/or different color flow imaging parameters may be used. Multiple parameters may be optimized simultaneously, such as by adjusting the values of the parameters in each iteration.

FIG. 2 shows an example sequence, example setting of the optimization strategies based on patient-specific target characteristic, and example setting of the values. In FIG. 2, the transmit frequency is set based on depth of the target. Target depth is used to set the transmit frequency. For deeper primary targets, the transmit frequency is reduced. For shallower primary targets, the transmit frequency is increased.

The depth of the target is used to select in acts 15A and 15B whether to increase or decrease the transmit frequency. For deep targets, the transmit frequency is set by decreasing by a given amount in act 16A. For shallow targets, the transmit frequency is set by increasing by a given amount in act 16B. The initial value may be maintained where the target is in a mid-depth range. In alternative embodiments, there is no initial or default value. Instead, the depth maps to specific values, such as 3.5 MHz for 5 cm and 2.5 MHz for 9 cm.

Based on previously acquired color flow data and/or based on acquiring additional color flow data with the transmit frequency as adjusted, the scale and/or gain is set in acts 16C and 16D using the criterion selected in act 15C. The value of the scale and/or gain are set using different optimization strategies for different levels of one or more characteristics. In the example of FIG. 2, the size (e.g., diameter) is used as the characteristic to select the optimization strategy in act 15C for setting the scale and/or the gain in acts 16C and 16D.

In act 15C, the selection for scale is between using an alias-based optimization or a flash-based optimization. The selection is based on size but may be based on pulsatility or both size and pulsatility. For example, the criterion for the scale parameter is aliasing when the size is above a threshold and flash when the size is below the threshold.

For large targets, the selected optimization may start with a small scale to induce aliasing and increase the scale until aliasing ceases to set the scale in act 16C. A large primary target biases optimization towards increasing the scale until there is no aliasing. Any measure may be used to check for aliasing, such as distribution of velocity values where no or few velocities occur at the edge parts (i.e., maximum positive and maximum negative) of the scale or distribution without little or no high gradients in velocity. A histogram may be used to determine aliasing.

For small targets, the selected optimization may start with a larger scale and decrease the scale in a way to reduce flash in act 16D. The scale is decreased until a level of flow is detected without flash. Flash may be measured as a threshold amount of deviation of average velocity by frame in the target or region of interest from a temporal average. Small target optimization emphasizes sensitivity, so the scale may be decreased until a certain level of flow is visible without flash. Decreasing scale increases the likelihood of flash, so flash measurement is used to find the smallest scale without flash. Where no target is detected, the same or different optimization may be used for scale (i.e., emphasize sensitivity).

In act 15C, the selection for gain is between using a bleeding-based optimization or a signal-to-noise ratio-based optimization. The selection is based on size but may be based on pulsatility or both size and pulsatility. For example, the criterion for the gain parameter is bleeding when the size is above a threshold and signal-to-noise ratio when the size is below the threshold.

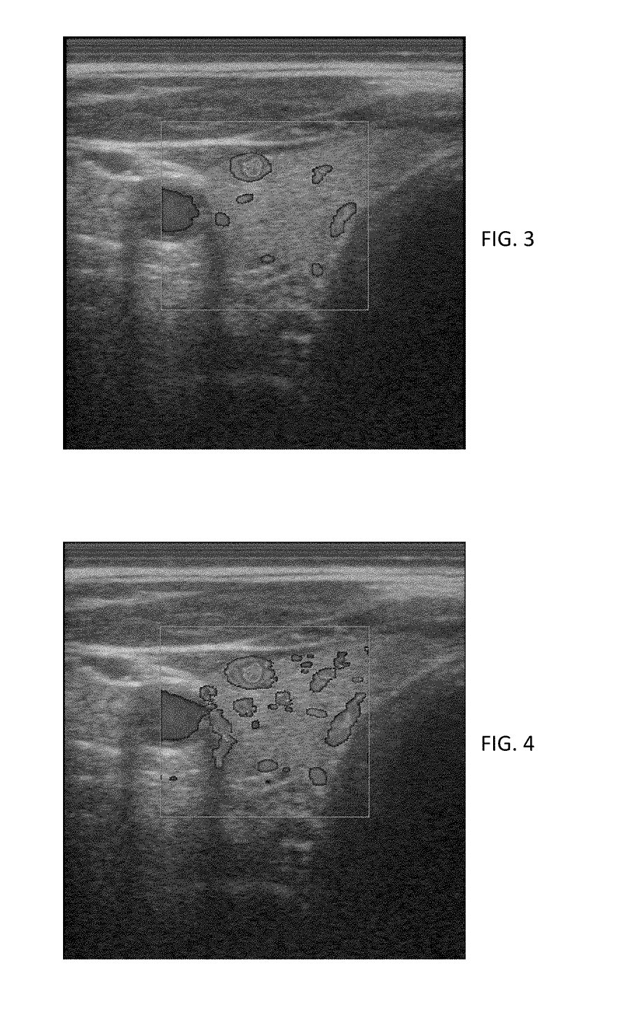

For large targets, the gain is adjusted to avoid bleed in act 16C. The gain is increased until bleed occurs or is adjusted from causing bleed to a level where the bleeding does not occur. The spatial extent of the target is known based on the segmentation. Where flow is detected as connected to but outside the boundaries of the target, then bleed is occurring. The optimization is to maximize the gain while avoiding bleed from too much gain. The gain is adjusted until the vessel, as identified in the segmentation, is filled in but not “bleeding over.” FIGS. 3 and 4 show example B-mode/color flow images of a thyroid. A lower gain is used in FIG. 3 than in FIG. 4. At high gain (e.g., FIG. 4), many more small vessels in the thyroid can be seen, but large vessels “bleed” over the vessel walls. For large primary targets (e.g., the large vessels), the gain is adjusted to fill the vessel but without bleed. Large vessels are identified in one image, and then the optimization prevents the large vessels from being over-gained as the gain is increased to expose the smaller vessels, thus preventing “bleeding” and simultaneously improving small vessel detectability.

For small targets, the gain is adjusted to provide a desired level of the signal-to-noise ratio in act 16D. The gain is increased to find a signal-to-noise ratio above a threshold level. The signal-to-noise ratio may be measured based on a noise frame acquired with no transmission as compared to a frame acquired using transmitted ultrasound. A ratio in average velocity or power of the two is the signal-to-noise ratio. Other measures of noise or the ratio may be used. At low gain, large vessels are well contained within the vessel boundaries, but the smaller vessels in the thyroid cannot be seen. For small primary targets or not visible targets, the gain is increased to find a sufficient signal-to-noise ratio. Using the signal-to-noise ratio emphasizes sensitivity in optimization for small targets or no targets like testes, breast, MSK, etc. imaging applications.

The gain may be set differently for different locations. The same optimization may be used for different locations, resulting in different gain values being provided for the different locations or targets. Alternatively, different optimization strategies are selected for the different locations, resulting in different gain values being provided for the different locations. The gain applied at different targets is the same or different, providing a gain map or mask. The gain is optimized by location.

In act 15D, the selection for persistence is between using a decrease or increase in persistence to set the value of persistence in acts 16E and 16F, respectively. The selection is based on pulsatility but may be based on size or both size and pulsatility. The criterion for the persistence may be the same, but the direction of change is different depending on the pulsatility. The optimization strategy is selected as increasing or decreasing persistence based on comparison of the pulastility to a threshold.

For large and/or highly pulsatile targets, the persistence is decreased. The level of persistence is adjusted downward until a level is achieved that is proportional to the duration of the pulsatility. Alternatively, the persistence is set to a given low level, such as no persistence. For small and/or low pulsatility targets, the persistence is increased until a level is achieved that is proportional to the duration of the pulsatility. Alternatively, the persistence is set to a given level based on a direct mapping from the pulsatility. Different persistence levels may be applied during different phases of the cardiac cycle.

Returning to FIG. 1, the ultrasound scanner performs color flow imaging of fluid or tissue in act 17. The imaging is configured by the values set based on the optimization. For example, the values for transmit frequency, scale, gain or gains, and/or persistence are used in color flow imaging.

The optimization may be on-going. As the color flow imaging is performed, feedback is provided to continue to adjust the value or values of one or more parameters. Alternatively, the values, once optimized, are used without further optimization for scanning the same region of interest or field of view for the patient.

For color flow imaging, an estimator or detector generates color flow data representing locations in the patient. Color flow data includes estimates of velocity, energy (e.g., power), and/or variance. The color flow data may be for fluid or tissue. Estimates of velocity, energy, and/or variance of tissue motion may be generated. Any motion data, whether from fluid or tissue movement, may be acquired.

The received spatial samples may be clutter filtered. The clutter filter passes frequencies associated with fluid and not tissue motion or with tissue motion and not fluid. The clutter filtering is of signals in a pulse sequence for estimating motion at a given time (e.g., samples of a flow sample count). A given signal may be used for estimates representing different times, such as associated with a moving window for clutter filtering and estimation. Different filter outputs are used to estimate motion for a location at different times.

Color flow data is generated from the spatial samples. Doppler processing, such as autocorrelation, may be used. In other embodiments, temporal correlation may be used. Another process may be used to estimate the color flow data. Color Doppler parameter values (e.g., velocity, energy, or variance values) are estimated from the spatial samples acquired at different times. The change in frequency (e.g., Doppler shift) between two samples for the same location at different times indicates the velocity. A sequence (flow sample count) of two or more samples may be used to estimate the color Doppler parameter values. Estimates are formed for different groupings of received signals, such as completely separate or independent groupings or overlapping groupings. The estimates for each grouping represent the spatial location at a given time.

The estimation is performed for the different sampled spatial locations. For example, velocities for the different locations in a plane are estimated from echoes responsive to the scanning. Multiple frames of color flow data may be acquired to represent the region of interest at different times, respectively.

The estimates may be thresholded. Thresholds are applied to the velocities and/or powers. For example, a low velocity threshold is applied. Velocities below the threshold are removed or set to another value, such as zero. As another example, where the energy is below a threshold, the velocity value for the same spatial location is removed or set to another value, such as zero. Alternatively, the estimated velocities are used without thresholding.

The acquired motion or color flow data is a frame of data or image representing the patient at a given time, despite being estimated from received signals over the flow sample count. The frames from different times may be temporally filtered, such as using an infinite or a finite impulse response.

Other data may be generated, such as B-mode data. A B-mode image may be overlaid with or have an incorporated region of interest showing the color Doppler velocities. Within the region of interest, locations with no flow are shown as B-mode data.

In act 18, the color flow image is displayed. The ultrasound system processes the frame of color flow data to create the image. Spatial filtering, temporal filtering, scan conversion, or another image processing is performed. The scalar values are mapped to display values, such as mapping to color values using a velocity scale. The resulting image is buffered for display. The display values are provided from the buffer to the display.

Color flow (e.g., Doppler energy or Doppler velocity), Doppler tissue motion, or other motion image is generated. The image may include other information. For example, the image is an overlay of the color flow data on B-mode data. For non-tissue locations or locations associated with sufficient flow, the color flow data (e.g., velocities) are used to determine a color to display. For tissue locations or low/no flow locations, the B-mode data is used.

Due to the optimization, the quality of the color flow imaging may be better than if default values or user-set values were used. The diagnostic information is increased by the optimization. Rather than using a default optimization, different strategies based on the scan of the patient or a priori information for the patient result in the image containing more diagnostically useful information and/or less artifacts, aliasing, and/or bleeding.

FIG. 5 shows one embodiment of a system 50 for optimizing flow imaging. The system 50 implements the method of FIG. 1, the method of FIG. 2, or another method. By using segmentation for scanning of a patient, a priori information is available to select the optimization to be used. The optimization is tuned to the patient and/or field of view to provide optimization appropriate for the given scan.

The system 50 includes a transmit beamformer 52, a transducer 53, a receive beamformer 54, a memory 55, a filter 56, a flow estimator 57, another memory 59, an image processor 58, a display 60, and a B-mode detector. Additional, different or fewer components may be provided. For example, the flow estimator 57 and image processor 58 are provided without the front-end components, such as the transmit and receive beamformers 12, 16. In yet another example, the memories 18 and 28 are one component. In one embodiment, the system 50 is a medical diagnostic ultrasound system. In an alternative embodiment, the system 50 is a computer or workstation. In yet another embodiment, the flow estimator 57 is part of a medical diagnostic ultrasound system or other medical imaging system, and the image processor 58 is part of a separate workstation or remote system, making of the ultrasound imaging system.

The transducer 53 is an array of a plurality of elements. The elements are piezoelectric or capacitive membrane elements. The array is configured as a one-dimensional array, a two-dimensional array, a 1.5D array, a 1.25D array, a 1.75D array, an annular array, a multidimensional array, a wobbler array, combinations thereof, or any other now known or later developed array. The transducer elements transduce between acoustic and electric energies. The transducer 53 connects with the transmit beamformer 52 and the receive beamformer 54 through a transmit/receive switch, but separate connections may be used in other embodiments.

The transmit and receive beamformers 52, 54 are a beamformer for scanning a region of the patient with the transducer 53. The transmit beamformer 52, using the transducer 53, transmits one or more beams to scan a region. Vector®, sector, linear or other scan formats may be used. The receive lines and/or transmit beams are distributed in the scan region. The receive beamformer 54 samples the receive beams at different depths. Sampling the same locations at different times obtains a sequence for flow estimation.

The transmit beamformer 52 is a processor, delay, filter, waveform generator, memory, phase rotator, digital-to-analog converter, amplifier, combinations thereof or any other now known or later developed transmit beamformer components. In one embodiment, the transmit beamformer 52 digitally generates envelope samples. Using filtering, delays, phase rotation, digital-to-analog conversion, and amplification, the desired transmit waveform is generated. Other waveform generators may be used, such as switching pulsers or waveform memories.

The transmit beamformer 52 is configured as a plurality of channels for generating electrical signals of a transmit waveform for each element of a transmit aperture on the transducer 53. The waveforms are unipolar, bipolar, stepped, sinusoidal or other waveforms of a desired center frequency or frequency band with one, multiple, and/or fractional number of cycles. The waveforms have relative delay and/or phasing and amplitude for focusing the acoustic energy. The transmit beamformer 52 includes a controller for altering an aperture (e.g. the number of active elements), an apodization profile (e.g., type or center of mass) across the plurality of channels, a delay profile across the plurality of channels, a phase profile across the plurality of channels, center frequency, frequency band, waveform shape, number of cycles and/or combinations thereof. A transmit beam focus is generated based on these beamforming parameters.

The receive beamformer 54 is a preamplifier, filter, phase rotator, delay, summer, base band filter, processor, buffers, memory, combinations thereof or other now known or later developed receive beamformer components. The receive beamformer 54 is configured into a plurality of channels for receiving electrical signals representing echoes or acoustic energy impinging on the transducer 53. A channel from each of the elements of the receive aperture within the transducer 53 connects to an amplifier and/or delay. An analog-to-digital converter digitizes the amplified echo signal. The digital radio frequency received data is demodulated to a base band frequency. Any receive delays, such as dynamic receive delays, and/or phase rotations are then applied by the amplifier and/or delay. A digital or analog summer combines data from different channels of the receive aperture to form return samples for one or a plurality of receive beams. The summer is a single summer or cascaded summer. In one embodiment, the beamform summer is configured to sum in-phase and quadrature channel data in a complex manner such that phase information is maintained for the formed beam. Alternatively, the beamform summer sums data amplitudes or intensities without maintaining the phase information.

The receive beamformer 54 is configured to form receive beams in response to the transmit beams. For example, the receive beamformer 54 receives one, two, or more receive beams in response to each transmit beam. The receive beams are collinear, parallel and offset or nonparallel with the corresponding transmit beams. The receive beamformer 54 outputs spatial samples representing different spatial locations of a scanned region. Once the channel data is beamformed or otherwise combined to represent spatial locations along the scan lines 51, the data is converted from the channel domain to the image data domain. The phase rotators, delays, and/or summers may be repeated for parallel receive beamformation. One or more of the parallel receive beamformers may share parts of channels, such as sharing initial amplification.

For B-mode scanning, the intensity of the echo for each location is determined by the B-mode detector 62. The field of view is scanned by sampling each location once to provide a frame of B-mode data for a given time.

For imaging motion, such as tissue motion or fluid motion, multiple transmissions and corresponding receptions are performed for each of a plurality of substantially same spatial locations. Phase changes between the different receive events for each given location indicate the velocity of the tissue or fluid. A velocity sample group corresponds to multiple transmissions for each of a plurality of scan lines 51. The number of times a substantially same spatial location, such as a scan line 51, is scanned within a velocity sample group is the velocity or flow sample count. The transmissions for different scan lines 51, different velocity sample groupings or different types of imaging may be interleaved. The amount of time between transmissions to a substantially same scan line 51 within the velocity sample count is the pulse repetition interval. The pulse repetition interval establishes the pulse repetition frequency or vice versa.

The memory 55 is video random-access memory, random access memory, removable media (e.g. diskette or compact disc), hard drive, database, corner turning memory, or other memory device for storing data or video information. In one embodiment, the memory 55 is a corner turning memory of a motion parameter estimation path. The memory 55 is configured to store signals responsive to multiple transmissions along a substantially same scan line. The memory 22 is configured to store ultrasound data formatted in an acoustic grid, a Cartesian grid, both a Cartesian coordinate grid and an acoustic grid, or ultrasound data representing a volume in a 3D grid. The return samples of the flow sample count for each of a plurality of locations are stored.

The filter 56 is a clutter filter, finite impulse response filter, infinite impulse response filter, analog filter, digital filter, combinations thereof or other now known or later developed filter. In one embodiment, the filter 56 includes a mixer to shift signals to baseband and a programmable low pass filter response for removing or minimizing information at frequencies away from the baseband. In other embodiments, the filter 56 is a low pass, high pass, or band pass filter. The filter 56 reduces velocities from fluids or alternatively reduces the influence of data from tissue while maintaining velocity information from fluids. The filter 56 has a set response or may be programmed, such as altering operation as a function of signal feedback or other adaptive process. In yet another embodiment, the memory 55 and/or the filter 56 are part of the flow estimator 57.

The Doppler or flow estimator 57 is a Doppler processor or cross-correlation processor for estimating the color data. In alternative embodiments, another device now known or later developed for estimating velocity, power (e.g., energy), and/or variance from any or various input data may be provided. The flow estimator 57 receives a plurality of signals associated with a substantially same location at different times and estimates a Doppler shift frequency, based on a change or an average change in phase between consecutive signals from the same location. Velocity is calculated from the Doppler shift frequency. Alternatively, the Doppler shift frequency is used as a velocity. The power and variance may also be calculated.

Color data (e.g., velocity, power, and/or variance) is estimated for spatial locations in the scan region from the beamformed scan samples. For example, the color data represents a plurality of different locations in a plane. The color flow data is motion data for tissue and/or fluid.

The flow estimator 57 may apply one or more thresholds to identify sufficient motion information. For example, velocity and/or power thresholding for identifying velocities is used. In alternative embodiments, a separate processor or filter applies thresholds. In other embodiments, the thresholding is applied after any motion suppression, such as by the image processor 58.

The flow estimator 57 outputs frames of data representing the scan region at different times. The beamformed samples for a given flow sample count are used to estimate for a time. A moving window with overlap of the data is used to estimate for other times. Velocities for each location at different times are output.

In one embodiment, the image processor 58 is or implements a filter, such as a temporal filter. Using the memory 59 as a buffer, a sequence of color flow data frames are filtered together providing persistence. The level or amount of persistence is programmable.

Alternatively or additionally, the image processor 58 is a digital signal processor, a general processor, an application specific integrated circuit, field programmable gate array, control processor, digital circuitry, analog circuitry, graphics processing unit, filter, combinations thereof or other now known or later developed device for optimizing the flow imaging. The image processor 58 operates pursuant to instruction provided in the memory 59, or a different memory for setting optimization approach and values based on the selected optimization approach. Additional or multiple processors may be used. The image processor 58 is configured by software, firmware, and/or hardware.

The image processor 58 receives B-mode and/or flow data from the B-mode detector, flow estimator 57, the memory 59, and/or another source. Using the received data, the image processor 58 is configured to identify color imaging targets in a region of interest and/or field of view. Segmentation is performed to identify the targets. Characteristics of the target or targets, such as the size, pulsatility, and/or location in the field of view, are detected from the segmentation. An optimization consideration, such as a criterion, measurement, strategy, approach, or other characteristic of optimization, is selected based on the characteristic of the target being scanned. A setting used in the configuration of color imaging is set based on the optimization consideration. The image processor 58 optimizes the setting using the selected consideration. For example, the size of the target is used to set the scale based on aliasing (large target) or flash (small target). As another example, the size of the target is used to set the gain based on color bleed (large target) or signal-to-noise ratio (small target). The image processor 58 selects the optimization to be used based on the characteristic (e.g., size) of the target or targets and then optimizes the setting using the selected optimization and feedback from the scanning for the patient.

The optimized settings configure the color imaging pipeline, such as the transmit beamformer 52, filter 56, estimator 57, persistence filter, and/or other components, for color imaging. The pipeline, including the estimator 57, is then used as configured by the image processor 58 to estimate motion values for color imaging. For example, flow values in a scan region or region of interest are acquired based on the configuration. The configuration is based on the characteristics of the target, so the color imaging configuration and resulting images is optimized based on the characteristics of the target for the patient.

The image processor 58 or other component uses the motion values to generate an image. The frame is scan converted and color mapped. The resulting color values are added to a B-mode image, such as an overlay, or used alone. The color values are placed in a display buffer to display an image on the display 60.

The display 60 is a CRT, LCD, plasma, projector, monitor, printer, touch screen, or other now known or later developed display device. The display 60 receives RGB, other color values, or other motion values and outputs an image. The image may be a gray scale or color image. The image represents the region of the patient with greater diagnostic information content due to the optimization based on segmented targets. The display 60 displays a Doppler or other color image from the motion values.

The memory 59 is video random-access memory, random access memory, removable media (e.g. diskette or compact disc), hard drive, database, or other memory device for storing color or other motion data. The stored data is in a polar or Cartesian coordinate format. The memory 59 is used by the image processor 58 for the various segmentation, prioritization, characteristic determination, optimization strategy selection, optimization, and/or configuration.

The instructions for implementing the processes, methods and/or techniques discussed above are provided on computer-readable storage media or memories, such as a cache, buffer, RAM, removable media, hard drive or other computer readable storage media. The memory 59 or other memory stores the instructions for optimization in Doppler imaging. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for optimizing color flow imaging by an ultrasound scanner, the method comprising:
scanning, by the ultrasound scanner, a patient;
segmenting a first color flow target represented in scan data from the scanning;
determining a size of the first color flow target;
setting a scale and/or gain using a different criterion depending on the size of the first color flow target;
color flow imaging, by the ultrasound scanner, fluid in the patient using the scale and/or gain.

2. The method of claim 1 wherein scanning comprises B-mode and/or B-mode and color scanning.

3. The method of claim 1 wherein segmenting comprises segmenting a boundary of the first color flow target as a vessel, and wherein determining the size comprises determining a diameter of the vessel.

4. The method of claim 1 wherein segmenting comprises segmenting the first color flow target and at least a second color flow target;
the method further comprising prioritizing the first and second color flow targets as a function of relative size, pulsatility, and/or location in a field of view of the scanning, wherein the first color flow target is assigned a greater priority such that the acts of determining and setting are based on the first color flow target.

5. The method of claim 1 wherein segmenting comprises segmenting the first color flow target and at least a second color flow target;
wherein setting comprises forming a gain mask as the gain, the gain mask including a different gain for the second color flow target than the gain for the first color flow target.

6. The method of claim 1 wherein setting comprises setting with the criterion for the scale being aliasing when the size is above a threshold and being flash when the size is below the threshold.

7. The method of claim 6 wherein setting comprises increasing the scale until aliasing ceases or decreasing the scale until a level of flow is detected without the flash.

8. The method of claim 1 wherein setting comprises setting with the criterion for the gain being bleed when the size is above a threshold and being signal-to-noise level when the size is below the threshold.

9. The method of claim 1 further comprising determining a depth of the first color flow target and setting a transmit frequency based on the depth.

10. The method of claim 1 further comprising determining a pulsatility of the first color flow target and setting a persistence based on the pulsatility.

11. A method for optimizing color flow imaging by an ultrasound scanner, the method comprising:
scanning, by the ultrasound scanner, a patient;
segmenting a first color flow target represented in scan data from the scanning;
setting a value for a color flow imaging parameter based on a first characteristic of the first color flow target as segmented; and
color flow imaging fluid in the patient using the value for the color flow imaging parameter.

12. The method of claim 11 wherein segmenting comprises segmenting a boundary of the first color flow target as a vessel, and wherein setting comprises setting based on the characteristic comprising a size, pulsatility, and/or location of the first color flow target.

13. The method of claim 11 wherein setting comprises setting the value using different optimization strategies for different levels of the first characteristic.

14. The method of claim 13 wherein the color flow imaging parameter comprises scale, the first characteristic comprises size, and wherein the different optimization strategies comprise alias or flash reduction based on the size being greater than or less than a threshold.

15. The method of claim 11 wherein the color flow imaging parameter comprises gain, the first characteristic comprises size, and wherein the different optimization strategies comprise optimization for bleeding or optimization for signal-to-noise based on the size being greater than or less than a threshold.

16. The method of claim 15 wherein setting comprises setting a gain mask with the value for the first color flow target and a different value for a second color flow target.

17. The method of claim 11 wherein the color flow imaging parameter comprises a transmit frequency, wherein the first characteristic comprises depth of the first color flow target, and wherein setting comprises setting the value for the transmit frequency based on the depth.

18. The method of claim 11 wherein the color flow imaging parameter comprises a persistence, wherein the first characteristic comprises a pulsatility of the first color flow target, and wherein setting comprises setting the value for the persistence based on the pulsatility.

19. An ultrasound system for optimizing color imaging, the ultrasound system comprising:
a transducer and beamformer for scanning a scan region;
a Doppler estimator configured to estimate, from the scanning, color values in the scan region, the configuration of the Doppler estimator being based on a detected size of a color region in the scan region; and
a display configured to display an image using the color values.

20. The ultrasound system of claim 19 further comprising an image processor configured to detect the size from the scanning and select a consideration for setting the configuration, the selection being based on the size such that a scale is set based on aliasing or flash and/or such that a gain is set based on color bleed or signal-to-noise ratio due to the size.
